(19) United States
(12) Patent Application Publication
(10) Pub. No.: US 2020/0297529 A1
(43) Pub. Date: Sep. 24, 2020

(54) SYSTEM FOR HYPOTHERMIA TREATMENT OF A PATIENT

(71) Applicant: Quickcool AB, Lund (SE)

(72) Inventors: Håkan PÅLSSON, Lund (SE); John WENNBORG, Lund (SE); Jonathan ODELDAHL, Ängelholm (SE)

(73) Assignee: Quickcool AB, Lund (SE)

(21) Appl. No.: 16/755,164

(22) PCT Filed: Apr. 24, 2018
(86) PCT No.: PCT/EP2018/060453

§ 371 Date: Apr. 10, 2020

(30) Foreign Application Priority Data
Oct. 11, 2017 (SE) 1751261-7
Jan. 23, 2018 (SE) 1850069-4

(51) Int. Cl. A61F 7/12; A61M 16/06; A61M 19/00

(52) U.S. Cl. CPC A61F 7/123; A61F 2007/0006; A61M 19/00; A61M 16/0666

(57) ABSTRACT

The disclosure relates to a system (100) for a patient (10), the system comprising one or more devices (110a; 110b). Each of the one or more devices (110a; 110b) comprises a nasal cavity element (120a; 120b) adapted to be introduced through a nostril into the nasal cavity (12) of the patient (10), tubing (130a; 130b), fluidly connected to the nasal cavity element (120a; 120b), said tubing (130a, 130b) being arranged to provide a fluid to the nasal cavity element (120a; 120b), and a sleeve (140a; 140b) slidably attached to said nasal cavity element (120a; 120b) and said tubing (130a; 130b). The sleeve (140a; 140b) is movable from a closed position (FIG. 2A) at which the sleeve (140a; 140b) encloses and protects the nasal cavity element (120a; 120b), and an open position (FIGS. 2B, 3B) at which at least a part of the nasal cavity element (120a; 120b) protrudes from the sleeve (140a; 140b).

### FIELD OF THE INVENTION

The present invention relates to a system for hypothermia treatment of a patient.

### BACKGROUND ART

Systems for hypothermia treatment are known in the art. Placing catheters or tubes in or via the nasal cavity are known procedures in intensive care. The approach may be advantageous as it leaves the mouth free for treatment or for ventilation of the patient. One kind of cooling catheter is a balloon catheter, which may be inflated when in its intended position, such as the nasal cavity, whereby the heat conduction to the surrounding tissue will be made more efficient. Balloon catheters inserted into the nasal cavity can be used for different purposes. One application is to transfer energy to the surrounding tissue in therapeutic hypothermia treatment. A problem of the art with this kind of systems for hypothermia treatment is that it is difficult to insert the catheters into the nasal cavity of the patient without the catheters being contaminated in the process. Contamination may occur both during storage of the catheter just prior to insertion, and due to the insertion process itself, for example by the balloon catheter accidently touching an exterior part of the patient, such as a part of the face, or a contamination present on the medical personnel.

### SUMMARY

It is an object to mitigate, alleviate or eliminate one or more of the above-identified deficiencies in the art and disadvantages singly or in any combination and solve at least the above mentioned problem.

According to a first aspect there is provided a system for a patient, the system comprising one or more devices, each of the one or more devices comprising:

a nasal cavity element, being a patient cooling element, adapted to be introduced through a nostril into the nasal cavity of the patent,

tubing, fluidly connected to the nasal cavity element, said tubing being arranged to provide a fluid to the nasal cavity element,

a sleeve slidably attached to said nasal cavity element and said tubing,

said sleeve being movable from a closed position at which the sleeve encloses and protects the nasal cavity element, and an open position at which at least a part of the nasal cavity element protrudes from the sleeve.

The proposed system may be advantageous as it may allow an improved resistance to contamination during handling and insertion into the nasal cavity prior to hypothermia treatment. By placing the nasal cavity element just outside a nostril of the patient, with the sleeve in its closed position thus covering and protecting it, the medical personnel may insert the nasal cavity element via the nose into the nasal cavity by holding the sleeve in a stable position in relation to the head of the patient using one hand, and gently sliding the nasal cavity element followed by the tubing connected to it into the nostril of the patient using the other hand. During the process, the sleeve will act both as a guide for inserting the nasal cavity element, and as a protective cover allowing to keep the nasal cavity element free from contamination. Furthermore, if the nasal cavity element is a balloon catheter, the sleeve will aid in keeping the balloon in a correct position during the insertion process so as to allow it to expand without any folding inside the nasal cavity.

According to some embodiments, the sleeve is a tube surrounding the nasal cavity element and the tubing.

According to some embodiments, the sleeve comprises a plastic material. The dimensions of the sleeve may vary dependent on the type of tubing used. According to some embodiments, an inner diameter of the sleeve is 3 times larger than an outer diameter of the tubing.

According to some embodiments, the first and second tube is arranged in parallel and in attachment with each other. The first and second tube may be made of a flexible material. According to some embodiments, the first and second tube is made of a plastic material, the first and second tube being attached to each other by welding.

According to some embodiments, the nasal cavity element is a luminary catheter, such as a balloon catheter, arranged to be expandable inside the nasal cavity of the patient.

According to some embodiments, the system further comprises a positioning strap being adapted to be fixated around the head of the patient, said positioning strap comprising:

one or more fixation elements adapted to fixate said tubing to the positioning strap in a respective fixation point, said one or more fixation elements being pivotable around said respective fixation point.

The positioning strap may be advantageous as it allows keeping the tubing fixed in relation to the head of the patient, once the nasal cavity element have been correctly inserted into the nasal cavity. That said, the one or more fixation elements being pivotable allows for a more flexible and comfortable fixation which is beneficial for both the patient and the medical personnel.

According to some embodiments, each of the one or more fixation elements comprises at least one recess for holding and fixating tubing therein. The at least one recess may be curved. This may be an advantageous solution as it provides a way to fixate tubing to a fixation element merely by disposing the tubing inside the recess, thus avoiding the need for any additional locking mechanisms. The curved recess provides a way to fixate the tubing to the fixation element purely by frictional forces between the tubing posterior and the inner surfaces of the at least one recess. The curvature of the recess may vary dependent on the tubing used. According to some embodiments, the radius of the curvature of the at least one recess is less than 3 times a width of the recess.

According to some embodiments, the at least one recess is tapered such that a width of the recess at the bottom thereof is smaller than a width of the recess at a top thereof. This implies that side walls of the recess will have an inclination, or alternatively, that opposing side walls of the recess will be un-parallel and form an angle with each other. The use of a tapered recess may increase the effectiveness of fixation to the fixation element as the tubing will be squeezed and kept in place by frictional forces. The inclination of the side walls of the recess may be different for different types of tubing. According to some embodiments, the angle between the opposing side walls is 22°.

According to some embodiments, the one or more fixation elements are removably attached to the positioning strap by means of a respective push button. This may be an advantageous solution as it allows replacing worn out fixation elements. Also, it allows the positioning strap to be custom-built for a specific use. Thus, for some embodiments, the positioning strap may have only one fixation element for fixating the tubing of two catheters. In other embodiments, the positioning strap may have four fixation elements, wherein each fixation elements holds one tube of the tubing.

According to some embodiments, the system comprises two devices, the nasal cavity elements of which are each adapted to be introduced through a respective nostril of the patient.

According to some embodiments, the one or more fixation elements are two fixation elements fixated to a respective fixation point distanced from each other along a circumferential direction of the strap, and wherein each fixation element being adapted to fixate tubing of a respective one from the two devices in said respective fixation point.

According to a second aspect there is provided a positioning strap for fixating a system for hypothermia treatment of a patient, said positioning strap being adapted to be fixated around the head of the patient, said positioning strap comprising:

one or more fixation elements adapted to fixate tubing of the system to the positioning strap in a respective fixation point, said one or more fixation elements being pivotable around said respective fixation point.

According to some embodiments, each of the one or more fixation elements comprises at least one recess for holding and fixating tubing therein.

According to some embodiments, the at least one recess is curved.

According to some embodiments, the radius of the curvature of the at least one recess is less than 3 times a width of the recess.

According to some embodiments, the at least one recess is tapered such that a width of the recess at the bottom thereof is smaller than a width of the recess at a top thereof.

According to some embodiments, the one or more fixation elements are two fixation elements fixated to a respective fixation point distanced from each other along a circumferential direction of the strap.

According to some embodiments, the one or more fixation elements are removably attached to the positioning strap by means of a respective push button.

### BRIEF DESCRIPTIONS OF THE DRAWINGS

The invention will by way of example be described in more detail with reference to the appended [schematic] drawings, which shows presently preferred embodiments of the invention.

FIG. 1 shows a cross sectional view of a system for hypothermia treatment of a patient according to embodiments of the present disclosure.

FIG. 2A shows a perspective view of a system for hypothermia treatment of a patient according embodiments of the present disclosure just prior to inserting the nasal cavity member into the nostril of the patient. Here the sleeve is in its closed position.

FIG. 2B shows a perspective view of a system for hypothermia treatment of a patient according embodiments of the present disclosure during insertion of the nasal cavity member through the nostril of the patient. Here, the sleeve is in its open position.

FIG. 3A shows a perspective view of a positioning strap positioned around the head of a patient according to embodiments of the present disclosure.

FIG. 3B shows a perspective view of the positioning strap of FIG. 3A when holding tubing of a device.

FIG. 4A shows a perspective view of a fixation element according to embodiments of the present disclosure.

FIG. 4B shows a cross sectional view of the fixating element along the line 4B-4B shown in FIG. 4A.

FIG. 5 shows a perspective top view of a system and a positioning strap according to alternative embodiments of the disclosure.

### DETAILED DESCRIPTION

The present invention will now be described more fully hereinafter with reference to the accompanying drawings, in which currently preferred embodiments of the invention are shown. This invention may, however, be embodied in many different forms and should not be construed as limited to the embodiments set forth herein; rather, these embodiments are provided for thoroughness and completeness, and fully convey the scope of the invention to the skilled person.

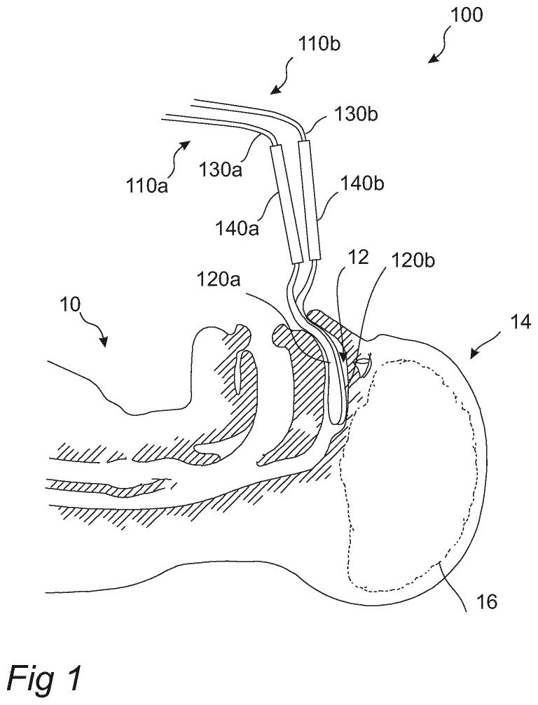

FIG. 1 shows a cross sectional view of a system 100 for hypothermia treatment of a patient 10. The purpose of the system 100 is to provide temperature control to at least parts of the body of the patient 10. Specifically, for the embodiment, the system 100 provides cooling to the brain 16 of the patient 10 via the nasal cavity 12. The system 100 comprises one or more devices 110a, 110b, in the form of cooling devices, each of the one or more devices 110a, 110b comprising a nasal cavity element 120a, 120b adapted to be introduced through a nostril into the nasal cavity 12 of the patent 10. Specifically, for the example embodiment, the system 100 comprises two devices 110a, 110b, the nasal cavity elements 120a, 120b of which are each adapted to be introduced through a respective nostril of the patient 10. In the example, the nasal cavity elements 120a, 120b are luminary catheters such as balloon catheters arranged to be expandable inside the nasal cavity 12 of the patient 10. The balloon catheters 120a, 120b are expanded by means of pressurizing a cooling fluid present within the balloon catheters 120a, 120b.

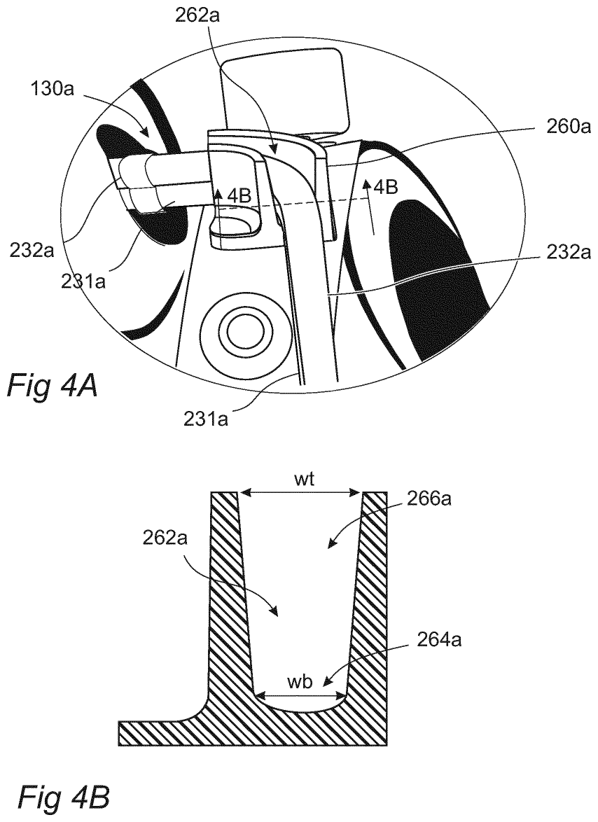

The cooling fluid is supplied to the balloon catheters 120a, 120b by means of tubing 130a, 130b which are fluidly connected to the nasal cavity element 120a, 120b. In FIG. 1, only one tube is shown for increased clearness. However, as illustrated in FIG. 4A, typically the tubing 130a, 130b comprises a first tube 231a, 231b for allowing the cooling fluid to enter the nasal cavity element 120a, 120b, and a second tube 232a, 232b for allowing the cooling fluid to leave the nasal cavity element 120a, 120b. The first 231a, 231b and second 232a, 232b tube is, for each tubing 130a, 130b of the example embodiment of FIG. 4A, arranged in parallel and in attachment with each other, thus forming a single supply unit for both inlet and outlet of cooling fluid to each balloon catheter 120a, 120b. However, alternatively, the first 231a, 231b and second 232a, 232b tube may be separate from each other. In such a case, the system may comprise four separate tubes, two inlet tubes and two outlet tubes. The tubing 130a, 130b is connected to an external heat regulating system comprising heat regulatory units and pumps. The heat regulating system is not explicitly disclosed herein. It should be understood that many alternative ways exist for achieving such a heat regulating system within the scope of the claims.

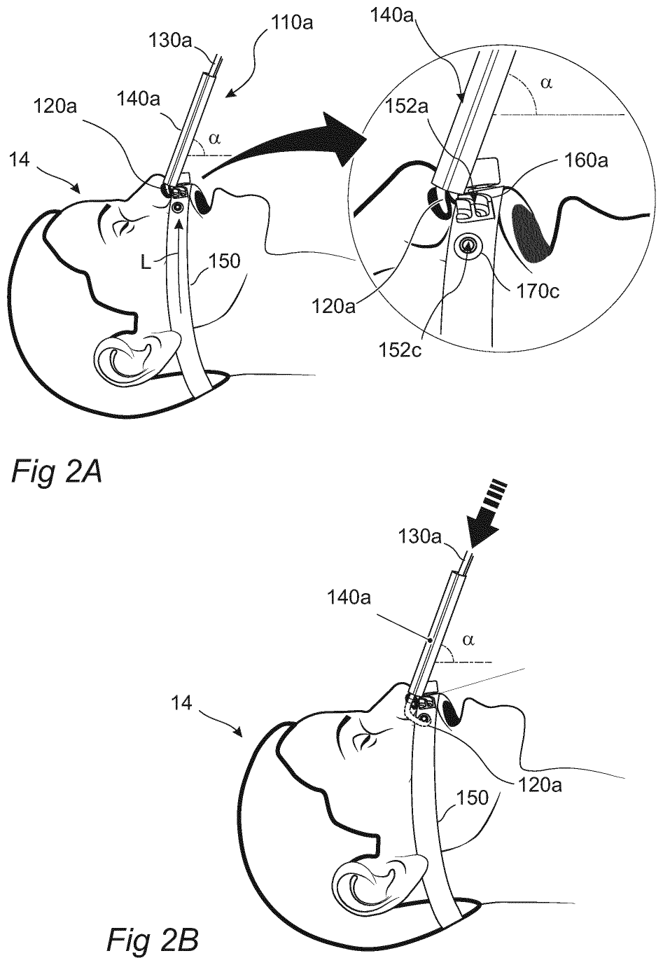

Each device of the system 100 further comprises a sleeve 140a, 140b slidably attached to the balloon catheter 120a, 120b and the tubing 130a, 130b. The sleeve 140a, 140b is movable from a closed position A at which the sleeve 140a, 140b encloses and protects the nasal cavity element 120a, 120b, and an open position B at which at least a part of the nasal cavity element 120a, 120b protrudes from the sleeve 140a, 140b. This is illustrated in FIG. 2A showing the sleeve in its closed position A just prior to inserting a balloon catheter into a nostril of the patient 10. Thus, the medical personnel may insert each balloon catheter 120a, 120b via the nose into the nasal cavity 12 by holding the respective sleeve 140a, 140b in a stable position in relation to the head 14 of the patient 10 using one hand, and gently sliding the balloon catheter 120a, 120b followed by the tubing 130a, 130b connected to it into the respective nostril of the patient 10 using the other hand. During the insertion process, each sleeve 140a, 140b will act both as a guide for inserting the respective balloon catheter 120a, 120b, and as a protective cover allowing to keep the respective balloon catheter 120a, 120b free from contamination. Specifically, the sleeve 140a, 140b aids in defining the direction of insertion of the balloon catheter 120a, 120b in relation to the head 14 of the patient 10, as illustrated in FIG. 2A using the angle α. Typically, the angle α is within the interval 40-80°. As illustrated in FIGS. 2A and 2B, each sleeve 140a, 140b is a tube surrounding the respective balloon catheter 120a; 120b and tubing 130a, 130b. The inner diameter of the sleeve 140a, 140b is typically 2-3 larger than the diameter of a tube of the tubing 130a, 130b. The sleeve 140a; 140b comprises a plastic material.

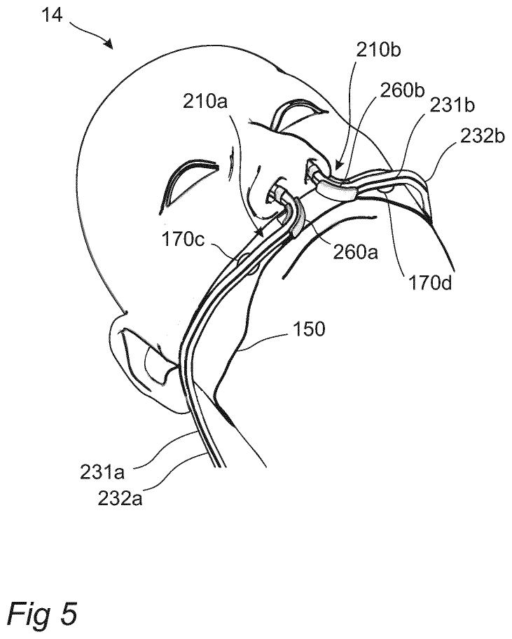

As illustrated in FIGS. 2A and 2B, the system 100 further comprises a positioning strap 150 being adapted to be fixated around the head 14 of the patient. The positioning strap 150 comprises one or more fixation elements 160a, 160b adapted to fixate the tubing 130a, 130b to the positioning strap 150 in a respective fixation point 152a, 152b. The one or more fixation elements 160a, 160b are pivotable around the respective fixation point 152a, 152b, thus allowing for a flexible and comfortable fixation which is beneficial for both the patient and the medical personnel. Specifically, the one or more fixation elements 160a, 160b are removably attached to the positioning strap 150 by means of a respective push button 170a, 170b. This provides a convenient solution where the number of fixation elements may be varied dependent on the task. Only one fixation element 160a is shown in FIGS. 2A and B. Another fixation element 160b may be fixated to the positioning strap 150 in the fixation point 152b on the other side of the nose. This is further illustrated in FIG. 5.

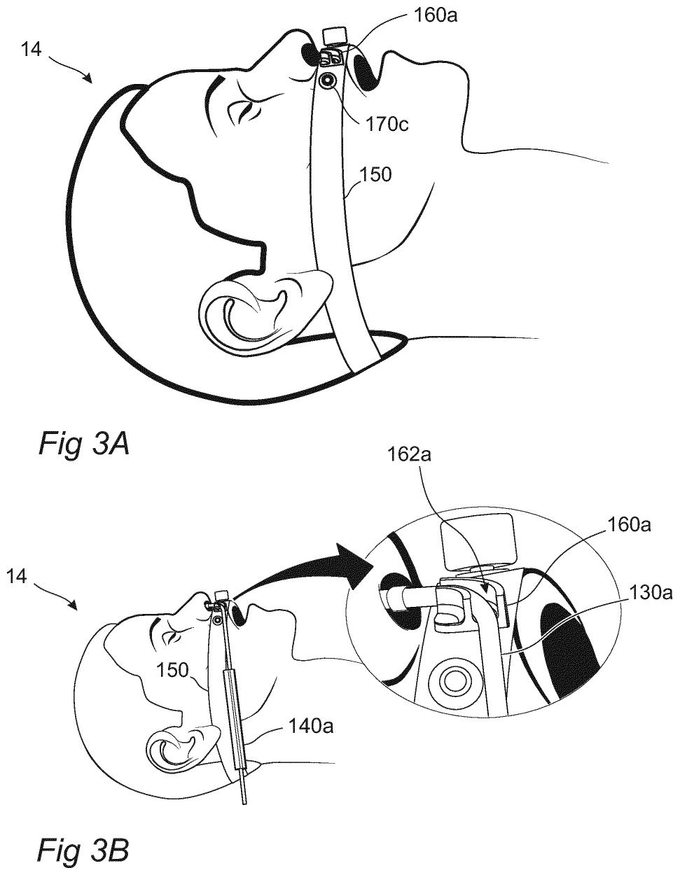

Each of the one or more fixation elements 160a, 160b comprises at least one recess 162a for holding and fixating tubing 130a, 130b therein. An example embodiment of the fixation element 160a comprising a single recess 162a is illustrated in FIGS. 3A and 3B. As most clearly seen in FIG. 3B, the recess 162a is curved. The radius of the curvature of the recess 162a is less than 3 times a width of the recess 162a. The curved recess 162a provides a way to fixate the tubing 130a to the fixation element purely by frictional forces between the tubing posterior and the inner surfaces of the recess 162a.

In the example embodiment shown in FIGS. 3A and 3B, the fixation element 160a is arranged to fixate one tube only. Thus, if two devices 110a, 110b are used where each device 110a, 110b being connected to two tubes, altogether four fixation elements would be needed. In an alternative example embodiment, a fixation element may be arranged to fixate more than one tube. This is illustrated in FIGS. 4A and 4B showing a fixation element 260a arranged to fixate two tubes, in this case an inlet and outlet tube for the balloon catheter 120a. Specifically, the tubing 130a comprises a first tube 231a for allowing the cooling fluid to enter the nasal cavity element 120a, and a second tube 232a for allowing the cooling fluid to leave the nasal cavity element 120a. The first 231a and second 232a tube is in the example embodiment of FIG. 4A arranged in parallel and in attachment with each other, thus forming a single supply unit for both inlet and outlet of cooling fluid to the balloon catheter 120a. As most clearly seen in the cross sectional view in FIG. 4B, the at least one recess 262a is tapered such that a width wb of the recess 262a at the bottom 264a thereof is smaller than a width wt of the recess 262a at a top 266a thereof.

As previously mentioned, the number of fixation elements may be varied dependent on the task and/or the example embodiment. According to one example embodiment, the one or more fixation elements are two fixation elements 160a, 160b fixated to a respective fixation point 152a, 152b distanced from each other along a circumferential direction L of the strap 150. This is best illustrated in FIG. 5 showing a perspective top view of a system 200 using two devices 210a and 210b fixated to the positioning strap 150 by the first fixation element 260a on one side of the nose and the second fixation element 260b on the other side of the nose, respectively. The other fixation element 160b may be fixated onto the button 170b (not shown in FIG. 5) or, alternatively, on another button (e.g. the button 170c or 170d) available on the positioning strap 150. Specifically, for the example embodiment of the positioning strap shown in FIG. 5, there are four buttons, 170a-d, of which the buttons 170a and 170b are used for fixating the fixation elements 160a and 160b, respectively, whereas the buttons 170c and 170d are not used. Each fixation element 160a, 160b is adapted to fixate tubing 130a, 130b of a respective one from the two devices 110a, 110b in said respective fixation point 152a, 152b.

The person skilled in the art realizes that the present invention by no means is limited to the preferred embodiments described above. On the contrary, many modifications and variations are possible within the scope of the appended claims.

For example, the tubing may be fixated to the fixation elements in other ways than disclosed herein. For example, a locking mechanism, such as a clamp may be used.

Additionally, variations to the disclosed embodiments can be understood and effected by the skilled person in practicing the claimed invention, from a study of the drawings, the disclosure, and the appended claims.

1. A fixation element for a system for a patient, the system for hypothermia treatment comprising one or more devices, each of the one or more devices comprising:
a nasal cavity element adapted to be introduced through a nostril into the nasal cavity of the patient,
tubing, fluidly connected to the nasal cavity element, said tubing being arranged to provide a fluid to the nasal cavity element,
the system further comprising a positioning strap being adapted to be fixated around the head of the patient,
wherein the fixation element being adapted to fixate said tubing to the positioning strap in a respective fixation point, the fixation element is pivotable around said fixation point, the fixation element further comprises at least one recess for holding and fixating tubing therein and wherein the at least one recess is curved.

2.-12. (canceled)

13. The fixation element according to claim 1, wherein the at least one recess is tapered such that a width of the recess at the bottom thereof is smaller than a width of the recess at a top thereof.

14. The fixation element according to claim 13, wherein the inclination between opposing side walls of the recess is 22°

15. The fixation element according to claim 1, wherein the fixation element is removably attached to the positioning strap by means of a push button.

16. The fixation element according to claim 1, wherein the curvature of the recess is less than 3 times a width of the recess.

17. The fixation element according to claim 1, wherein two tubes can be fixated by the fixation element.

18. A positioning strap for a system for hypothermia treatment of a patient, the system for hypothermia treatment comprising one or more devices, each of the one or more devices comprising:
a nasal cavity element adapted to be introduced through a nostril into the nasal cavity of the patient,
tubing, fluidly connected to the nasal cavity element, said tubing being arranged to provide a fluid to the nasal cavity element,
wherein the positioning strap is adapted to be fixated around the head of the patient, the positioning strap further comprising one or more fixation elements according to claim 1 being adapted to fixate said tubing to the positioning strap in a respective fixation point.

19. The positioning strap according to claim 18, wherein the one or more fixation elements are removably attached to the positioning strap by means of a respective push button.

20. The positioning strap according to claim 18, wherein the one or more fixation elements are two fixation elements fixated to a respective fixation point distanced from each other along a circumferential direction of the strap, and wherein each fixation element being adapted to fixate tubing in said respective fixation point.

21. The positioning strap according to claim 18, wherein the positioning strap comprises four fixation elements, wherein each fixation elements holds one tube of the tubing.

22. The positioning strap according to claim 18, wherein the at least one recess is tapered such that a width of the recess at the bottom thereof is smaller than a width of the recess at a top thereof.

23. The positioning strap according to claim 18, wherein the fixation element is removably attached to the positioning strap by means of a push button.

24. The positioning strap according to claim 18, wherein the curvature of the recess is less than 3 times a width of the recess.

25. The positioning strap according to claim 18, wherein two tubes can be fixated by the fixation element.

26. A method for hypothermia treatment of a patient by means of a system for hypothermia treatment comprising one or more devices, each of the one or more devices comprising:
a nasal cavity element adapted to be introduced through a nostril into the nasal cavity of the patient,
tubing, fluidly connected to the nasal cavity element, said tubing being arranged to provide a fluid to the nasal cavity element,
the system further comprising a positioning strap, said positioning strap comprising one or more fixation elements, the fixation element being adapted to fixate said tubing to the positioning strap in a respective fixation point, the method comprising:
introducing the nasal cavity element through a nostril into the nasal cavity of the patient,
connecting the tubing to the nasal cavity element, said tubing being arranged to provide a fluid to the nasal cavity element,
fixating the positioning strap around the head of the patient and fixating said tubing to the one or more fixation elements of the positioning strap in said respective fixation point.

27. The method for hypothermia treatment of a patient according to claim 26, further comprising removably attaching the one or more fixation elements to the positioning strap by means of a respective push button.

28. The method for hypothermia treatment of a patient according to claim 26, wherein the fixation element further comprises at least one recess for holding and fixating tubing therein and wherein the at least one recess is curved, and wherein the at least one recess is tapered such that a width of the recess at the bottom thereof is smaller than a width of the recess at a top thereof, wherein the fixating of said tubing to the one or more fixation elements comprises fixating said tubing to the one or more fixation elements purely by frictional forces.

29. The method for hypothermia treatment of a patient according to claim 26, wherein the method further comprises removably attaching the fixation element to the positioning strap by means of a push button.

30. The method for hypothermia treatment of a patient according to claim 28, wherein the curvature of the recess is less than 3 times a width of the recess and wherein the fixating of said tubing to the one or more fixation elements comprises fixating said tubing to the one or more fixation elements purely by frictional forces.

31. The method for hypothermia treatment of a patient according to claim 26, wherein fixating said tubing to the one or more fixation elements comprises fixating two tubes to the one or more fixation element.
